(19) United States
(12) Patent Application Publication
(10) Pub. No.: US 2019/0269585 A1
(43) Pub. Date: Sep. 5, 2019

(54) THICKENING AND ELONGATION OF KERATINOUS FIBERS

(71) Applicant: Coty Inc., New York, NY (US)

(72) Inventors: Patricia Haro, Hackensack, NJ (US); Dhavai Patel, Baskin Ridge, NJ (US); Rossana Lanza, Glen Rock, NJ (US); Vibha K. Shah, Edison, NJ (US)

(21) Appl. No.: 16/301,928

(22) PCT Filed: Jun. 13, 2017
(86) PCT No.: PCT/US2017/037250

§ 371 Date: Nov. 15, 2018

Related U.S. Application Data
(60) Provisional application No. 62/349,289, filed on Jun. 13, 2016.

(51) Int. Cl. A61K 8/19; A61K 8/81; A61K 8/88; A61K 8/73; A61K 8/55; A61Q 1/10; A61K 8/92

(52) U.S. Cl. CPC A61K 8/19; A61K 8/8152; A61K 8/88; A61K 8/731; A61K 2800/43; A61Q 1/10; A61K 8/8176; A61K 8/92; A61K 8/553

(57) ABSTRACT

Inventive embodiments include a formulation, that includes at least one pigment encapsulated by a transparent, unbreakable and non-absorbing material; at least one elongating agent; at least two adhesive agents; at least one continuous phase viscosity modifying agent; and at least one or more waxes selected from a group that includes naturally derived and synthetic waxes, hard and soft, in a concentration 10-20% weight.

This application claims the benefit of priority to U.S. Provisional Patent Application Ser. No. 62/349,289 titled “THICKENING AND ELONGATION OF KERATINOUS FIBERS,” filed Jun. 13, 2016, the disclosure of which is incorporated herein in its entirety by reference.

### FIELD

Inventive subject matter disclosed herein relates to formulation embodiments for dressing and treating hair such as eyebrow and eyelash hair.

### BACKGROUND

False eyelashes, such as eyelash and hair extensions, and semi-permanent hair dressings have long been used to make up eye brows and eye lashes. However, false eyelashes and extensions for dressing hair are expensive, time consuming, and irritating. Additionally, some consumers have lost false eyelashes while performing tasks such as dressing.

Multi-step products have been developed to impart an appearance to users that mimic the look imparted by false lashes and extensions, but with fewer invasive application procedures. Unfortunately for consumers, the multi-step products failed to provide the fake lashes and extensions look without undesirable side effects. Prior art single step products have not been able to provide a desired makeup result.

Additionally, prior art products are time consuming to apply to hair and lashes and brows. Prior art products are also messy, clumpy, dull looking, and/or flake during application and during wear of the product. Furthermore, at least some of the existing products are irritating because fibers fall into eyes of the consumers. Existing products containing high levels of fibers also tend to be tacky and/or get very dry over time.

### SUMMARY

Inventive embodiments include a hair dressing formulation, that includes at least one organic and/or inorganic pigment encapsulated by a transparent, unbreakable and non-absorbing material; at least one elongating agent, which is premade for some embodiments and created in situ for other embodiments. The formulation also includes at least two adhesive agents; at least one continuous phase viscosity modifying agent; and at least one or more waxes selected from a group that includes naturally derived and synthetic waxes, hard and soft, in a concentration of 10-20% by weight.

Inventive embodiments disclosed herein also include a hair dressing formulation. One formulation includes the following ingredients:

| Ingredient | Wet Wt. % |
|---|---|
| Stage 1 | |
| CARNAUBA WAX PRIME YELLOW 2442 | 1%-2% |
| CANDELILLA WAX 2039Y | 0.25%-1.25% |
| 8104 KAHL WHITE BEESWAX | 4%-6% |
| OZOKERITE WAX 7726 | 0.25%-0.75% |
| STEARIC ACID COS | 2.5%-4% |
| PARACERA P | 4%-6% |
| LEXGARD O | 0.25%-0.75% |
| UNIMER U-15 | 1%-3% |
| LEXOREZ 200 | 0.5%-0.75% |
| Stage 2 | |
| BENTONE GEL CAO V | 1.5%-2.1% |
| Stage 3 | |
| BLACK IRON OXIDE C33-5000 | 2%-4% |
| BLACK IRON OXIDE C33-6000 | 5%-7% |
| TALC EXTRA 5/0 DEC | 0.05%-0.15% |
| SWD7550 BLACK IRON OXIDE SYNTHETIC | 0.5%-1.2% |
| WAX DISP | |
| Stage 4 | |
| PVP K-30 | 0.25%-0.75% |
| Stage 5 | |
| SILICONE ANTIEM SE2 | 0.05%-0.15% |
| Stage 6 | |
| CELL U LASH 150 | 0.1%-0.2% |
| NFBL-10D-2T | 0.1%-0.2% |
| NFCB 17D 4T | 0.05%-0.2% |
| Stage 7 | |
| WATER, DEIONIZED | 40%-50% |
| ARGININE | 1.4%-2.0% |
| TWEEN 40 | 1 4%-2 0% |
| PVP K-30 | 0.25%-0.75% |
| LANETTE 16 | 1.4%-1.7% |
| SYNCROWAX HR-C-Tribehenic | 1.10%-1.20% |
| STEARIC ACID COS | 1.8%-2.5% |
| SILICONE ANTIEM SE2 | 0.05%-.015% |
| PHENOXYETHANOL | 0.3%-0.9% |
| EMULMETIK 100J | 0.10%-0.50% |
| Stage 8 | |
| PROPYLENE GLYCOL | 2%-6% |
| WATER, DEIONIZED | 1%-6% |
| D-PANTHENOL 75L | 1.1%-1.5% |
| VITAMIN E ACETATE | 0.05%-0.2% |
| MICROMER C60/42 | 1%-2% |
| DC 749 FLUID | 0.1%-0.9% |
| CARBONCAP 20 | 1%-2% |

Another embodiment includes the following formulation:

| Ingredient | Wet Wt. % |
|---|---|
| Cera Carnauba/Copernicia Cerifera (Carnauba) | 1.3%-1.9% |
| Euphorbia Cerifera (Candelilla)Wax | 1.90%-1.4% |
| Beeswax | 4.5%-6% |
| Paraffin and Microcrystalline Wax and Polyethylene | 1%-1.5% |
| Stearic Acid | 3%-4.6% |
| Paraffin | 5%-7.5% |
| Caprylyl Glycol | 0.25%-0.75% |
| VP/Eicosene Gopolymer | 1.8%-2.8% |
| Tris (Nonylphenyl) Phosphite (and) Adipic Acid (and) | 1%-2% |
| PHASE B | |
| Propylene Carbonte(ans) Stearalkonium Hectorite | 1.8%-2.6% |
| PHASE C | |
| Iron Oxides | 2%-3.5% |
| Iron Oxides | 4.5%-7% |
| Talc | 0.05%-0.75% |
| Black Iron Oxides and Synthetic Wax | 0.8%-2.1% |
| PHASE D | |
| PVP | 0.05%-0.2% |
| PHASE E | |
| Water and Simethicone | 0.05%-0.2% |
| PHASE F | |
| Water | 40%-60% |
| Arginine | 1%-3% |
| Polysorbate 40 | 1%-3% |
| PVP | 0.05%-0.2% |
| Cetyl alcohol | 1.7%-2.3% |
| Tribehenin | 1%-2% |
| Stearic Acid | 2%-3.1% |
| Water (and) Simethicone | 0.05%-0.2% |
| Phenoxyethanol | 0.1%-0.9% |
| Lecithin | 0.05%-0.2% |
| PHASE G | |
| Cellulose | 0.05%-0.2% |
| Nylon 6 (and) Iron Oxides (and) Triethoxycaprylylsilane | 0.05%-0.2% |
| Nylon 6 and Black 2 | 0.01%-0.1% |
| PHASE H | |
| Propylene Glycol | 2%-4% |
| Water | 1%-10% |
| Water (and) Panthenol (and0 Pantolactone | 0.5%-1.2% |
| Tocopheryl Acetate | 0.01%-0.1% |
| Ammonium Acrylates Copolymer (and) Water (and) | 0.5%-1% |
| Cyclopentasiloxane (and) trimethylsiloxysilicate | 0.1%-0.6% |
| D&C Black No 2 (and) Polymethyl Methacrylate (and) | 0.4%-1% |

Another embodiment includes the following formulation:

| Ingredient | Wet Wt. % |
|---|---|
| PHASE A | |
| CERA CARNAUBA/COPERNICIA CERIFERA | 0.00%-20.00% |
| (CARNAUBA) WAX/CIRE DE CARNAUBA | |
| EUPHORBIA CERIFERA (CANDELILLA) WAX | 0.00%-20.00% |
| BEESWAX | 0.00%-20.00% |
| PARAFFN | 0.00%-20.00% |
| MICROCRSTALLINE WAX | |
| POLYETHYLENE | |
| SYNTHETIC WAX | |
| STEARIC ACID | 0.00%-20.00% |
| PARAFFIN | 0.00%-20.00% |
| CAPRYLYL GLYCOL | 0.00%-0.511% |
| VP/EICOSENE COPOLYMER | 0.00%-5.00% |
| TRIS(NONYLPHENYL)PHOSPHITE | 0.00%-3.00% |
| ADIPIC ACID | |
| GLYCERIN | |
| TRIMETHYLPENTANEDIOL/ADIPIC ACID/ | |
| GLYCERIN CROSSPOLYMER | |
| PHASE B | |
| PROPYLENE CARBONATE | 0.00%-2.00% |
| STEARALKONIUM HECTORITE | |
| RICINUS COMMUNIS (CASTOR) SEED OIL | |
| PHASE C | |
| IRON OXIDES | 0.00%-15.00% |
| IRON OXIDES | 0.00%-15.00% |
| TALC | 0.00%-5.00% |
| BLACK IRON OXIDES | 0.00%-3.00% |
| SYNTHETIC WAX | |
| PHASE D | |
| PVP | 0.00%-4.00% |
| PHASE E | |
| WATER | 0.00%-0.30% |
| SIMETHICONE | |
| PHASE F | |
| WATER | QS |
| ARGININE | 0.00%-2.00% |
| POLYSORBATE 40 | 0.00%-2.50% |
| PVP | 0.00%-4.00% |
| CETYL ALCOHOL | 0.00%-20.00% |
| TRIBEHENIN | 0.00%-20.00% |
| STEARIC ACID | 0.00%-20.00% |
| WATER | 0.00%-0.30% |
| SIMETHICONE | |
| PHENOXYETHANOL | 0.00%-0.985% |
| LECITHIN | 0.00%-1.00% |
| PHASE G | |
| CELLULOSE | 0.00%-2.00% |
| NYLON-6 | 0.00%-2.00% |
| IRON OXIDES | |
| TRIETHOXYCAPRYLYLSILANE | |
| SILICA | |
| NYLON-6 and BLACK 2 | 0.00%-2.00% |
| PHASE H | |
| PROPYLENE GLYCOL | 0.00%-4.00% |
| WATER | QS |
| WATER | 0.00%-4.00% |
| PANTHENOL | |
| PANTOLACTONE | |
| TOCOPHERYL ACETATE | 0.00%-1.00% |
| AMMONIUM ACRYLATES COPOLYMER | 0.00-5.00% |
| WATER | |
| ALCOHOL DENAT. | |
| DISODIUM DECETH-6 SULFOSUCCINATE | |
| LAURETH-30 | |
| SODIUM DEHYDROACETATE | |
| CYCLOPENTASILOXANE | 0.00%-3.00% |
| TRIMETHYLSILOXYSILICATE | |
| D&C BLACK NO. 2 | 0.00%-3.00% |
| POLYMETHYL METHACRYLATE | |
| BUTYLENE GLYCOL COCOATE | |

### DETAILED DESCRIPTION

The following detailed description includes “examples,” that are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense, and the scope of the present invention is defined by the appended claims and their equivalents.

In this document, the terms “a” or “an” are used to include one or more than one and the term “or” is used to refer to a nonexclusive “or” unless otherwise indicated. In addition, it is to be understood that the phraseology or terminology employed herein, and not otherwise defined, is for the purpose of description only and not of limitation. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

As used herein, the term, “hair dressing” refers to a coating for one or more of an eye lash or one or more supercilium of an eyebrow.

Hair dressing formulation embodiments disclosed herein deliver buildable volume and elongation to hair strands and lashes giving a full lash fringe effect that is aimed to mimic fake lashes and hair extensions. Furthermore, the hair dressing formulation embodiments are delivered to lashes in a one easy step similar to delivery of traditional mascaras.

Inventive embodiments herein include a cosmetic composition that includes a multi-particle dispersion and emulsion system with at least one organic and/or inorganic pigment, encapsulated by a transparent, unbreakable and non-absorbent material, at least one elongating agent which is premade and/or created in situ, at least two adhesive agents, at least one continuous phase viscosity modifying agent, and at least one or more waxes selected from a group that includes naturally derived and synthetic waxes, hard and soft waxes, in a concentration of 10-20% by weight.

inventive hair dressing formulation embodiments disclosed herein include particles, such as black iron oxide particles. In one embodiment, the black iron oxide particles are C33-5000 and C33-6000, manufactured by Sun Chemical Corp. At least some of the particles include encapsulated pigment enclosed within strong, transparent, non-absorbent and unbreakable shells. One embodiment of these encapsulated particles are SWD7550 Black Iron Oxide Synthetic Wax DISP, manufactured by Sun Chemical Corp. Some particle embodiments also include talc particles, such as Talc Extra 5/0 DEC, manufactured by Imerys Minerals. Another embodiment includes Carbon Black enrobed with CarbonCap 20, manufactured by TAGRA.

The hair dressing formulation embodiments also include elongating materials that are premade for some embodiments or for other embodiments, are formed in situ. The formulation is built up on strands of hair and lashes to which it is applied by cellulose and nylon fibers such as those listed in Stage 6 above. All of these fiber ingredients are manufactured by Kobo

Products. It has surprisingly been found that when fibers are formed in situ, a larger concentration of fibers is possible in the formulation without adversely impacting thickening and elongation of a user's eyelashes. Conventional mascara employing fibers that are not formed in situ do not display the long wear, thickening and elongation of lashes displayed by the mascara formulation embodiments disclosed herein.

Emulsifiers include liquid soybean lecithin, such as EMULMETIK 100J, manufactured by Lucas Meyer Cosmetics.

Rheological control and particle suspension in an emulsion are provided by the Bentone Gel Cao V ingredient, manufactured by Elementis Specialties. Tribehenin, sold as Syncrowax HRX, manufactured by Croda, is added to modify properties of other waxes by softening structure and reducing crystallinity and rigidity.

Film forming polymers, also acting as adhesives in the formulation include vinyl pyrrolidone/Eicosene copolymer, one formulation of which is UNIMER U-15, manufactured by induchem companies and polyvinyl pyrrolidone, having the trade name, PVP K-30, manufactured by Ashland, Inc. Another film forming polymer employed is a Trimethylpentanediol/Adipic Acid/Glycerin Crosspolymer sold under the tradename, LEXOREZ 200 by

Hair dressing formulation embodiments also include waxes such as Carnauba wax Prime Yellow 2442, manufactured by Kahl GmbH and Co., Candelilla Wax 2039Y, manufactured by Kahl Wax Refinery in Trittau, DE, 8104 Kahl White Beeswax, also manufactured by Kahl Wax Refinery in Trittau, DE, Ozokerite Wax 7726, manufactured by Poth Hille & Co. and Paracera P-Wax, manufactured by Paramelt.

These formulation ingredients provide consumers with a single step hair dressing application for ease of use. The formulation embodiments also impart a significantly enhanced thickness and elongation effect which existing and prior art products are not able to achieve.

The inventive formulation disclosed herein provide a hair dressing that delivers buildable volume and elongation of hair, thus imparting a full lash fringe effect that is aimed to mimic fake lashes and hair extensions. Furthermore, formulation embodiments disclosed herein are delivered to lashes in a one easy step similar to the application of conventional mascaras.

One formulation embodiment is as follows:

| Ingredient | Wet Wt. % |
|---|---|
| Stage 1 | |
| CARNAUBA WAX PRIME YELLOW 2442 | 1%-2% |
| CANDELILLA WAX 2039Y | 0.25%-1.25% |
| 8104 KAHL WHITE BEESWAX | 4%-6% |
| OZOKERITE WAX 7726 | 0.25%-0.75% |
| STEARIC ACID COS | 2.5%-4% |
| PARACERA P | 4%-6% |
| LEXGARD O | 0.25%-0.75% |
| UNIMER U-15 | 1%-3% |
| LEXOREZ 200 | 0.5%-0.75% |
| Stage 2 | |
| BENTONE GEL CAO V | 1.5%-2.1% |
| Stage 3 | |
| BLACK IRON OXIDE C33-5000 | 2%-4% |
| BLACK IRON OXIDE C33-6000 | 5%-7% |
| TALC EXTRA 5/0 DEC | 0.05%-0.15% |
| SWD7550 BLACK IRON OXIDE SYNTHETIC | 0.5%-1.2% |
| WAX DISP | |
| Stage 4 | |
| PVP K-30 | 0.25%-0.75% |
| Stage 5 | |
| SILICONE ANTIEM SE2 | 0.05%-0.15% |
| Stage 6 | |
| CELL U LASH 150 | 0.1%-0.2% |
| NFBL-10D-2T | 0.1%-0.2% |
| NFCB 17D 4T | 0.05%-0.2% |
| Stage 7 | |
| WATER, DEIONIZED | 40%-50% |
| ARGININE | 1.4%-2.0% |
| TWEEN 40 | 1.4%-2.0% |
| PVP K-30 | 0.25%-0.75% |
| LANETTE 16 | 1.4%-1.7% |
| SYNCROWAX HR-C-Tribehenic | 1.10%-1.20% |
| STEARIC ACID COS | 1.8%-2.5% |
| SILICONE ANTIEM SE2 | 0.05%-.015% |
| PHENOXYETHANOL | 0.3%-0.9% |
| EMULMETIK 100J | 0.10%-0.50% |
| Stage 8 | |
| PROPYLENE GLYCOL | 2%-6% |
| WATER, DEIONIZED | 1%-6% |
| D-PANTHENOL 75L | 1.1%-1.5% |
| VITAMIN E ACETATE | 0.05%-0.2% |
| MICROMER C60/42 | 1%-2% |
| DC 749 FLUID | 0.1%-0.9% |
| CARBONCAP 20 | 1%-2% |

In one embodiment, at least a portion of the organic and/or inorganic pigment of Stage 3 is encapsulated by a transparent, unbreakable and non-absorbent material, SWD7550 BLACK IRON OXIDE SYNTHETIC WAX DISP, manufactured by Sun Chemical Corp.

The combination of these ingredients imparts a flexible and buildable lash product, uniformity in intensity of the pigments, and continuous smooth layers of elongated film even after product has (hied. The long lasting film of formula adheres onto the lashes to provide zero flaking or smudging effects. The formulation embodiments impart a multi-dispersion/emulsion system that provides ease of the formulation to be both flexible and buildable with the help of the emulsifiers in both the water and oil phases. It does so by being a water-oil-water emulsion that provides long lasting wear, while still being able to provide all the added benefits of the formula being creamy, buildable, with great volume and length.

The addition of Polyvinyl Pyrrolidone, in Stage 4 of the formulation is added to water stage 7. Vinyl pyrrolidone/Eicosene copolymer is also included in an oil phase in Stage 1. Thus, Polyvinyl Pyrrolidone is present in both an oil and a water phase. An acrylates copolymer in Stage 8, Micromer C60/42, manufactured by Worlee, allows for durable film formation that 2.0 prevents the flaking and smudging that often times plagues a user using conventional mascara. The PVP also adds gloss, color intensity and due to its tacky feel it secures the lashes for a stay put application.

The multifunction unbreakable encapsulated pigment particles aid in intense and uniform color development, non-drying, volumizing and lengthening benefits.

A moisturizer, D-Panthenol 75L, manufactured by Coptis is added as a moisturizer in some embodiments.

For Some embodiments, Procapil, manufactured by Sederma, is Butylene Glycol (and) Aqua (and) PPG-26-Buteth-26 (and) PEG-40 Hydrogenated Castor Oil (and) Apigenin (and) Oleanolic Acid (and) Biotinoyl Tripeptide-1 and is added to prevent hair loss. The term, “QS” used to describe the concentration of water refers to the amount of water added to take the formulation to 100 percent by weight.

Another formulation embodiment includes the following:

| Ingredient | Wet Wt. % |
|---|---|
| Cera Carnauba/Copernicia Cerifera (Carnauba) | 1.3%-1.9% |
| Euphorbia Cerifera (Candelilla)Wax | 1.90%-1.4% |
| Beeswax | 4.5%-6% |
| Paraffin and Microcrystalline Wax and Polyethylene | 1%-1.5% |
| Stearic Acid | 3%-4.6% |
| Paraffin | 5%-7.5% |
| Caprylyl Glycol | 0.25%-0.75% |
| VP/Eicosene Gopolymer | 1.8%-2.8% |
| ris (Nonylphenyl) Phosphite (and) Adipic Acid (and) | 1%-2% |
| Phase B | |
| Propylene Carbonte(ans) Stearalkonium Hectorite | 1.8%-2.6% |
| Phase C | |
| Iron Oxides | 2%-3.5% |
| Iron Oxides | 4.5%-7% |
| Talc | 0.05%-0.75% |
| Black Iron Oxides and Synthetic Wax | 0.8%-2.1% |
| PHASE D | |
| PVP | 0.05%-0.2% |
| PHASE E | |
| Water and Simethicone | 0.05%-0.2% |
| PHASE F | |
| Water | 40%-60% |
| Arginine | 1%-3% |
| Polysorbate 40 | 1%-3% |
| PVP | 0.05%-0.2% |
| Cetyl alcohol | 1.7%-2.3% |
| Tribehenin | 1%-2% |
| Stearic Acid | 2%-3.1% |
| Water (and) Simethicone | 0.05%-0.2% |
| Phenoxyethanol | 0.1%-0.9% |
| Lecithin | 0.05%-0.2% |
| PHASE G | |
| Cellulose | 0.05%-0.2% |
| Nylon 6 (and) Iron Oxides (and) Triethoxycaprylylsilane | 0.05%-0.2% |
| Nylon 6 and Black 2 | 0.01%-0.1% |
| PHASE H | |
| Propylene Glycol | 2%-4% |
| Water | 1%-10% |
| Water (and) Panthenol (and0 Pantolactone | 0.5%-1.2% |
| Tocopheryl Acetate | 0.01%-0.1% |
| Ammonium Acrylates Copolymer (and) Water (and) | 0.5%-1% |
| Cyclopentasiloxane (and) trimethylsiloxysilicate | 0.1%-0.6% |
| D&C Black No 2 (and) Polymethyl Methacrylate (and) | 0.4%-1% |

Another formulation includes the following formulation:

| Ingredient | Wet Wt. % |
|---|---|
| PHASE A | |
| CERA CARNAUBA/COPERNICIA CERIFERA | 0.00%-20.00% |
| (CARNAUBA) WAX/CIRE DE CARNAUBA | |
| EUPHORBIA CERIFERA (CANDELILLA) WAX | 0.00%-20.00% |
| BEESWAX | 0.00%-20.00% |
| PARAFFN | 0.00%-20.00% |
| MICROCRSTALLINE WAX | |
| POLYETHYLENE | |
| SYNTHETIC WAX | |
| STEARIC ACID | 0.00%-20.00% |
| PARAFFIN | 0.00%-20.00% |
| CAPRYLYL GLYCOL | 0.00%-0.511% |
| VP/EICOSENE COPOLYMER | 0.00%-5.00% |
| TRIS(NONYLPHENYL)PHOSPHITE | 0.00%-3.00% |
| ADIPIC ACID | |
| GLYCERIN | |
| TRIMETHYLPENTANEDIOL/ADIPIC ACID/ | |
| GLYCERIN CROSSPOLYMER | |
| PHASE B | |
| PROPYLENE CARBONATE | 0.00%-2.00% |
| STEARALKONIUM HECTORITE | |
| RICINUS COMMUNIS (CASTOR) SEED OIL | |
| PHASE C | |
| IRON OXIDES | 0.00%-15.00% |
| IRON OXIDES | 0.00%-15.00% |
| TALC | 0.00%-5.00% |
| BLACK IRON OXIDES | 0.00%-3.00% |
| SYNTHETIC WAX | |
| PHASE D | |
| PVP | 0.00%-4.00% |
| PHASE E | |
| WATER | 0.00%-0.30% |
| SIMETHICONE | |
| PHASE F | |
| WATER | QS |
| ARGININE | 0.00%-2.00% |
| POLYSORBATE 40 | 0.00%-2.50% |
| PVP | 0.00%-4.00% |
| CETYL ALCOHOL | 0.00%-20.00% |
| TRIBEHENIN | 0.00%-20.00% |
| STEARIC ACID | 0.00%-20.00% |
| WATER | 0.00%-0.30% |
| SIMETHICONE | |
| PHENOXYETHANOL | 0.00%-0.985% |
| LECITHIN | 0.00%-1.00% |
| PHASE G | |
| CELLULOSE | 0.00%-2.00% |
| NYLON-6 | 0.00%-2.00% |
| IRON OXIDES | |
| TRIETHOXYCAPRYLYLSILANE | |
| SILICA | |
| NYLON-6 and BLACK 2 | 0.00%-2.00% |
| PHASE H | |
| PROPYLENE GLYCOL | 0.00%-4.00% |
| WATER | QS |
| WATER | 0.00%-4.00% |
| PANTHENOL | |
| PANTOLACTONE | |
| TOCOPHERYL ACETATE | 0.00%-1.00% |
| AMMONIUM ACRYLATES COPOLYMER | 0.00-5.00% |
| WATER | |
| ALCOHOL DENAT. | |
| DISODIUM DECETH-6 SULFOSUCCINATE | |
| LAURETH-30 | |
| SODIUM DEHYDROACETATE | |
| CYCLOPENTASILOXANE | 0.00%-3.00% |
| TRIMETHYLSILOXYSILICATE | |
| D&C BLACK NO. 2 | 0.00%-3.00% |
| POLYMETHYL METHACRYLATE | |
| BUTYLENE GLYCOL COCOATE | |

### ADDITIONAL EMBODIMENTS

The following exemplary embodiments are provided, the numbering of which is not to be construed as designating levels of importance:

Embodiment 1 provides a formulation, comprising:

at least one pigment encapsulated by a transparent, unbreakable and non-absorbing material;

at least one elongating agent;

at least two adhesive agents;

at leak one continuous phase viscosity modifying agent; and

at least one or more waxes selected from a group that includes naturally derived and synthetic waxes, hard and soft, in a concentration of 10-20% by weight.

Embodiment 2 provides the formulation of Embodiment wherein the pigment is a black iron oxide.

Embodiment 3 provides the formulation according to any one of Embodiments 1 or 2, wherein the transparent, unbreakable and non-absorbing material is polymethyl methacrylate.

Embodiment 4 provides the formulation according to any one of Embodiments 1-3, wherein the elongation agent is soybean lecithin.

Embodiment 5 provides the formulation according to any one of Embodiments 1-3, wherein the elongation agent comprises cellulose and nylon fibers that include premade fibers and fibers that are formed in situ.

Embodiment 6 provides the formulation according to any one of Embodiments 1-4, wherein an adhesive comprises polyvinylpyrrolidone,

Embodiment 7 provides the formulation according to any one of Embodiments 1-5, wherein the continuous phase modifying agent is Bentone Gel CAO V.

Embodiment 8 provides a formulation consisting essentially of:

| Ingredient | Wet Wt. % |
|---|---|
| Stage 1 | |
| CARNAUBA WAX PRIME YELLOW 2442 | 1%-2% |
| CANDELILLA WAX 2039Y | 0.25%-1.25% |
| 8104 KAHL WHITE BEESWAX | 4%-6% |
| OZOKERITE WAX 7726 | 0.25%-0.75% |
| STEARIC ACID COS | 2.5%-4% |
| PARACERA P | 4%-6% |
| LEXGARD O | 0.25%-0.75% |
| UNIMER U-15 | 1%-3% |
| LEXOREZ 200 | 0.5%-0.75% |
| Stage 2 | |
| BENTONE GEL CAO V | 1.5%-2.1% |
| Stage 3 | |
| BLACK IRON OXIDE C33-5000 | 2%-4% |
| BLACK IRON OXIDE C33-6000 | 5%-7% |
| TALC EXTRA 5/0 DEC | 0.05%-0.15% |
| SWD7550 BLACK IRON OXIDE SYNTHETIC | 0.5%-1.2% |
| WAX DISP | |
| Stage 4 | |
| PVP K-30 | 0.25%-0.75% |
| Stage 5 | |
| SILICONE ANTIEM SE2 | 0.05%-0.15% |
| Stage 6 | |
| CELL U LASH 150 | 0.1%-0.2% |
| NFBL-10D-2T | 0.1%-0.2% |
| NFCB 17D 4T | 0.05%-0.2% |
| Stage 7 | |
| WATER, DEIONIZED | 40%-50% |
| ARGININE | 1.4%-2.0% |
| TWEEN 40 | 1.4%-2.0% |
| PVP K-30 | 0.25%-0.75% |
| LANETTE 16 | 1.4%-1.7% |
| SYNCROWAX HR-C-Tribehenic | 1.10%-1.20% |
| STEARIC ACID COS | 1.8%-2.5% |
| SILICONE ANTIEM SE2 | 0.05%-.015% |
| PHENOXYETHANOL | 0.3%-0.9% |
| EMULMETIK 100J | 0.10%-0.50% |
| Stage 8 | |
| PROPYLENE GLYCOL | 2%-6% |
| WATER, DEIONIZED | 1%-6% |
| D-PANTHENOL 75L | 1.1%-1.5% |
| VITAMIN E ACETATE | 0.05%-0.2% |
| MICROMER C60/42 | 1%-2% |
| DC 749 FLUID | 0.1%-0.9% |
| CARBONCAP 20 | 1%-2% |

Embodiment 9 provides a formulation, comprising:

| Ingredient | Wet Wt. % |
|---|---|
| Cera Carnauba/Copernicia Cerifera (Carnauba) | 1.3%-1.9% |
| Euphorbia Cerifera (Candelilla)Wax | 1.90%-1.4% |
| Beeswax | 4.5%-6% |
| Paraffin and Microcrystalline Wax and Polyethylene | 1%-1.5% |
| Stearic Acid | 3%-4.6% |
| Paraffin | 5%-7.5% |
| Caprylyl Glycol | 0.25%-0.75% |
| VP/Eicosene Gopolymer | 1.8%-2.8% |
| Tris (Nonylphenyl) Phosphite (and) Adipic Acid (and) | 1%-2% |
| PHASE B | |
| Propylene Carbonte(ans) Stearalkonium Hectorite | 1.8%-2.6% |
| PHASE C | |
| Iron Oxides | 2%-3.5% |
| Iron Oxides | 4.5%-7% |
| Talc | 0.05%-0.75% |
| Black Iron Oxides and Synthetic Wax | 0.8%-2.1% |
| PHASE D | |
| PVP | 0.05%-0.2% |
| PHASE E | |
| Water and Simethicone | 0.05%-0.2% |
| PHASE F | |
| Water | 40%-60% |
| Arginine | 1%-3% |
| Polysorbate 40 | 1%-3% |
| PVP | 0.05%-0.2% |
| Cetyl alcohol | 1.7%-2.3% |
| Tribehenin | 1%-2% |
| Stearic Acid | 2%-3.1% |
| Water (and) Simethicone | 0.05%-0.2% |
| Phenoxyethanol | 0.1%-0.9% |
| Lecithin | 0.05%-0.2% |
| PHASE G | |
| Cellulose | 0.05%-0.2% |
| Nylon 6 (and) Iron Oxides (and) Triethoxycaprylylsilane | 0.05%-0.2% |
| Nylon 6 and Black 2 | 0.01%-0.1% |
| PHASE H | |
| Propylene Glycol | 2%-4% |
| Water | 1%-10% |
| Water (and) Panthenol (and0 Pantolactone | 0.5%-1.2% |
| Tocopheryl Acetate | 0.01%-0.1% |
| Ammonium Acrylates Copolymer (and) Water (and) | 0.5%-1% |
| Cyclopentasiloxane (and) trimethylsiloxysilicate | 0.1%-0.6% |
| D&C Black No 2 (and) Polymethyl Methacrylate (and) | 0.4%-1% |

Embodiment 10 provides a formulation comprising:

| Ingredient | Wet Wt. % |
|---|---|
| PHASE A | |
| CERA CARNAUBA/COPERNICIA CERIFERA | 0.00%-20.00% |
| (CARNAUBA) WAX/CIRE DE CARNAUBA | |
| EUPHORBIA CERIFERA (CANDELILLA) WAX | 0.00%-20.00% |
| BEESWAX | 0.00%-20.00% |
| PARAFFN | 0.00%-20.00% |
| MICROCRSTALLINE WAX | |
| POLYETHYLENE | |
| SYNTHETIC WAX | |
| STEARIC ACID | 0.00%-20.00% |
| PARAFFIN | 0.00%-20.00% |
| CAPRYLYL GLYCOL | 0.00%-0.511% |
| VP/EICOSENE COPOLYMER | 0.00%-5.00% |
| TRIS(NONYLPHENYL)PHOSPHITE | 0.00%-3.00% |
| ADIPIC ACID | |
| GLYCERIN | |
| TRIMETHYLPENTANEDIOL/ADIPIC ACID/ | |
| GLYCERIN CROSSPOLYMER | |
| PHASE B | |
| PROPYLENE CARBONATE | 0.00%-2.00% |
| STEAKALKONIUM HECTORITE | |
| RICINUS COMMUNIS (CASTOR) SEED OIL | |
| PHASE C | |
| IRON OXIDES | 0.00%-15.00% |
| IRON OXIDES | 0.00%-15.00% |
| TALC | 0.00%-5.00% |
| BLACK IRON OXIDES | 0.00%-3.00% |
| SYNTHETIC WAX | |
| PHASE D | |
| PVP | 0.00%-4.00% |
| PHASE E | |
| WATER | 0.00%-0.30% |
| SIMETHICONE | |
| PHASE F | |
| WATER | QS |
| ARGININE | 0.00%-2.00% |
| POLYSORBATE 40 | 0.00%-2.50% |
| PVP | 0.00%-4.00% |
| CETYL ALCOHOL | 0.00%-20.00% |
| TRIBEHENIN | 0.00%-20.00% |
| STEARIC ACID | 0.00%-20.00% |
| WATER | 0.00%-0.30% |
| SIMETHICONE | |
| PHENOXYETHANOL | 0.00%-0.985% |
| LECITHIN | 0.00%-1.00% |
| PHASE G | |
| CELLULOSE | 0.00%-2.00% |
| NYLON-6 | 0.00%-2.00% |
| IRON OXIDES | |
| TRIETHOXYCAPRYLYLSILANE | |
| SILICA | |
| NYLON-6 and BLACK 2 | 0.00%-2.00% |
| PHASE H | |
| PROPYLENE GLYCOL | 0.00%-4.00% |
| WATER | QS |
| WATER | 0.00%-4.00% |
| PANTHENOL | |
| PANTOLACTONE | |
| TOCOPHERYL ACETATE | 0.00%-1.00% |
| AMMONIUM ACRYLATES COPOLYMER | 0.00-5.00% |
| WATER | |
| ALCOHOL DENAT. | |
| DISODIUM DECETH-6 SULFOSUCCINATE | |
| LAURETH-30 | |
| SODIUM DEHYDROACETATE | |
| CYCLOPENTASILOXANE | 0.00%-3.00% |
| TRIMETHYLSILOXYSILICATE | |
| D&C BLACK NO. 2 | 0.00%-3.00% |
| POLYMETHYL METHACRYLATE | |
| BUTYLENE GLYCOL COCOATE | |

The above mentioned technology and combination of raw materials work together synergistically to provide a unique formula that differs significantly from the existing fiber products out in the market.

The embodiments are described in sufficient detail to enable those skilled in the art to practice the invention. Other embodiments may be utilized and formulation and method of using changes may be made without departing from the scope of the invention. The detailed description is not to be taken in a limiting sense, and the scope of the invention is defined only by the appended claims, along with the full scope of equivalents to which such claims are entitled.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. A formulation, comprising:
at least one pigment encapsulated by a transparent, unbreakable and non-absorbing material;
at least one elongating agent;
at least two adhesive agents;
at least one continuous phase viscosity modifying agent; and
at least one or more waxes selected from a group that includes naturally derived and synthetic waxes, hard and soft, in a concentration of 10-20% by weight.

2. The formulation of claim 1, wherein the pigment is a black iron oxide.

3. The formulation according to claim 1, wherein the transparent, unbreakable and non-absorbing material is polymethyl methacrylate.

4. The formulation according to claim 1, wherein the elongation agent is soybean lecithin.

5. The formulation according to claim 1, wherein the elongation agent comprises cellulose and nylon fibers that include premade fibers and fibers that are formed in situ.

6. The formulation according to claim 1, wherein an adhesive comprises polyvinylpyrrolidone.

7. The formulation according to claim 1, wherein the continuous phase modifying agent is Bentone Gel CAO V.

8. A formulation consisting essentially of:

| Ingredient | Wet Wt. % |
|---|---|
| Stage 1 | |
| CARNAUBA WAX PRIME YELLOW 2442 | 1%-2% |
| CANDELILLA WAX 2039Y | 0.25%-1.25% |
| 8104 KAHL WHITE BEESWAX | 4%-6% |
| OZOKERITE WAX 7726 | 0.25%-0.75% |
| STEARIC ACID COS | 2.5%-4% |
| PARACERA P | 4%-6% |
| LEXGARD O | 0.25%-0.75% |
| UNIMER U-15 | 1%-3% |
| LEXOREZ 200 | 0.5%-0.75% |
| Stage 2 | |
| BENTONE GEL CAO V | 1.5%-2.1% |
| Stage 3 | |
| BLACK IRON OXIDE C33-5000 | 2%-4% |
| BLACK IRON OXIDE C33-6000 | 5%-7% |
| TALC EXTRA 5/0 DEC | 0.05%-0.15% |
| SWD7550 BLACK IRON OXIDE SYNTHETIC | 0.5%-1.2% |
| WAX DISP | |
| Stage 4 | |
| PVP K-30 | 0.25%-0.75% |
| Stage 5 | |
| SILICONE ANTIEM SE2 | 0.05%-0.15% |
| Stage 6 | |
| CELL U LASH 150 | 0.1%-0.2% |
| NFBL-10D-2T | 0.1%-0.2% |
| NFCB 17D 4T | 0.05%-0.2% |
| Stage 7 | |
| WATER, DEIONIZED | 40%-50% |
| ARGININE | 1.4%-2.0% |
| TWEEN 40 | 1.4%-2.0% |
| PVP K-30 | 0.25%-0.75% |
| LANETTE 16 | 1.4%-1.7% |
| SYNCROWAX HR-C-Tribehenic | 1.10%-1.20% |
| STEARIC ACID COS | 1.8%-2.5% |
| SILICONE ANTIEM SE2 | 0.05%-.015% |
| PHENOXYETHANOL | 0.3%-0.9% |
| EMULMETIK 100J | 0.10%-0.50% |
| Stage 8 | |
| PROPYLENE GLYCOL | 2%-6% |
| WATER, DEIONIZED | 1%-6% |
| D-PANTHENOL 75L | 1.1%-1.5% |
| VITAMIN E ACETATE | 0.05%-0.2% |
| MICROMER C60/42 | 1%-2% |
| DC 749 FLUID | 0.1%-0.9% |
| CARBONCAP 20 | 1%-2% |

9. A formulation comprising:

| Ingredient | Wet Wt. % |
|---|---|
| Cera Carnauba/Copernicia Cerifera (Carnauba) | 1.3%-1.9% |
| Euphorbia Cerifera (Candelilla)Wax | 1.90%-1.4% |
| Beeswax | 4.5%-6% |
| Paraffin and Microcrystalline Wax and Polyethylene | 1%-1.5% |
| Stearic Acid | 3%-4.6% |
| Paraffin | 5%-7.5% |
| Caprylyl Glycol | 0.25%-0.75% |
| VP/Eicosene Gopolymer | 1.8%-2.8% |
| Tris (Nonylphenyl) Phosphite (and) Adipic Acid (and) | 1%-2% |
| PHASE B | |
| Propylene Carbonte(ans) Stearalkonium Hectorite | 1.8%-2.6% |
| PHASE C | |
| Iron Oxides | 2%-3.5% |
| Iron Oxides | 4.5%-7% |
| Talc | 0.05%-0.75% |
| Black Iron Oxides and Synthetic Wax | 0.8%-2.1% |
| PHASE D | |
| PVP | 0.05%-0.2% |
| PHASE E | |
| Water and Simethicone | 0.05%-0.2% |
| PHASE F | |
| Water | 40%-60% |
| Arginine | 1%-3% |
| Polysorbate 40 | 1%-3% |
| PVP | 0.05%-0.2% |
| Cetyl alcohol | 1.7%-2.3% |
| Tribehenin | 1%-2% |
| Stearic Acid | 2%-3.1% |
| Water (and) Simethicone | 0.05%-0.2% |
| Phenoxyethanol | 0.1%-0.9% |
| Lecithin | 0.05%-0.2% |
| PHASE G | |
| Cellulose | 0.05%-0.2% |
| Nylon 6 (and) Iron Oxides (and) Triethoxycaprylylsilane | 0.05%-0.2% |
| Nylon 6 and Black 2 | 0.01%-0.1% |
| PHASE H | |
| Propylene Glycol | 2%-4% |
| Water | 1%-10% |
| Water (and) Panthenol (and0 Pantolactone | 0.5%-1.2% |
| Tocopheryl Acetate | 0.01%-0.1% |
| Ammonium Acrylates Copolymer (and) Water (and) | 0.5%-1% |
| Cyclopentasiloxane (and) trimethylsiloxysilicate | 0.1%-0.6% |
| D&C Black No 2 (and) Polymethyl Methacrylate (and) | 0.4%-1% |

10. A formulation comprising:

| Ingredient | Wet Wt. % |
|---|---|
| PHASE A | |
| CERA CARNAUBA/COPERNICIA CERIFERA | 0.00%-20.00% |
| (CARNAUBA) WAX/CIRE DE CARNAUBA | |
| EUPHORBIA CERIFERA (CANDELILLA) WAX | 0.00%-20.00% |
| BEESWAX | 0.00%-20.00% |
| PARAFFN | 0.00%-20.00% |
| MICROCRSTALLINE WAX | |
| POLYETHYLENE | |
| SYNTHETIC WAX | |
| STEARIC ACID | 0.00%-20.00% |
| PARAFFIN | 0.00%-20.00% |
| CAPRYLYL GLYCOL | 0.00%-0.511% |
| VP/EICOSENE COPOLYMER | 0.00%-5.00% |
| TRIS(NONYLPHENYL)PHOSPHITE | 0.00%-3.00% |
| ADIPIC ACID | |
| GLYCERIN | |
| TRIMETHYLPENTANEDIOL/ADIPIC ACID/ | |
| GLYCERIN CROSSPOLYMER | |
| PHASE B | |
| PROPYLENE CARBONATE | 0.00%-2.00% |
| STEARALKONIUM HECTORITE | |
| RICINUS COMMUNIS (CASTOR) SEED OIL | |
| PHASE C | |
| IRON OXIDES | 0.00%-15.00% |
| IRON OXIDES | 0.00%-15.00% |
| TALC | 0.00%-5.00% |
| BLACK IRON OXIDES | 0.00%-3.00% |
| SYNTHETIC WAX | |
| PHASE D | |
| PVP | 0.00%-4.00% |
| Phase E | |
| WATER | 0.00%-0.30% |
| SIMETHICONE | |
| PHASE F | |
| WATER | QS |
| ARGININE | 0.00%-2.00% |
| POLYSORBATE 40 | 0.00%-2.50% |
| PVP | 0.00%-4.00% |
| CETYL ALCOHOL | 0.00%-20.00% |
| TRIBEHENIN | 0.00%-20.00% |
| STEARIC ACID | 0.00%-20.00% |
| WATER | 0.00%-0.30% |
| SIMETHICONE | |
| PHENOXYETHANOL | 0.00%-0.985% |
| LECITHIN | 0.00%-1.00% |
| PHASE G | |
| CELLULOSE | 0.00%-2.00% |
| NYLON-6 | 0.00%-2.00% |
| IRON OXIDES | |
| TRIETHOXYCAPRYLYLSILANE | |
| SILICA | |
| NYLON-6 and BLACK 2 | 0.00%-2.00% |
| PHASE H | |
| PROPYLENE GLYCOL | 0.00%-4.00% |
| WATER | QS |
| WATER | 0.00%-4.00% |
| PANTHENOL | |
| PANTOLACTONE | |
| TOCOPHERYL ACETATE | 0.00%-1.00% |
| AMMONIUM ACRYLATES COPOLYMER | 0.00-5.00% |
| WATER | |
| ALCOHOL DENAT. | |
| DISODIUM DECETH-6 SULFOSUCCINATE | |
| LAURETH-30 | |
| SODIUM DEHYDROACETATE | |
| CYCLOPENTASILOXANE | 0.00%-3.00% |
| TRIMETHYLSILOXYSILICATE | |
| D&C BLACK NO. 2 | 0.00%-3.00% |
| POLYMETHYL METHACRYLATE | |
| BUTYLENE GLYCOL COCOATE | |
